(19) United States
(12) Patent Application Publication
(10) Pub. No.: US 2018/0185078 A1
(43) Pub. Date: Jul. 5, 2018

(54) INTER-LAMINAR VERTEBRAL IMPLANT APPARATUS AND METHODS OF IMPLANATION

(71) Applicant: Eos Biotech, Gangnam-Gu (KR)

(72) Inventors: Seon Joo Na, Songpa-Gu (KR); Jongwoo Kim, Sungnam-si (KR)

(21) Appl. No.: 15/396,811

(22) Filed: Jan. 2, 2017

(51) Int. Cl. A61B 17/88; A61B 17/70; A61B 17/68

(52) U.S. Cl. CPC A61B 17/88; A61B 17/1671; A61B 17/68; A61B 17/7062

(57) ABSTRACT

Embodiments of an inter-laminar vertebral implant apparatus for insertion and/or implantation between laminar of adjacent superior and inferior vertebrae sized and configured for implantation into such inter-laminar space, the apparatus configured to be located between adjacent laminar and including an engagement mechanism for operatively coupling the apparatus to the adjacent laminar and for preventing migration of the apparatus once implanted. Various inter-laminar implant apparatus configurations may be employed to conform to the anatomy of a patient's spine to be treated. Other embodiments may be described and claimed.

### TECHNICAL FIELD

Various embodiments described herein relate generally to treating adjacent bony structures, including apparatus and methods for creating and maintaining a desired spacing between adjacent bony structures.

### BACKGROUND INFORMATION

It may be desirable create and maintain a desired spacing between adjacent bony structure via an implant assembly. The present invention provides such an apparatus and method of implantation.

### BRIEF DESCRIPTION OF THE DRAWINGS

FIG. 1A is a simplified isometric diagram of a mammalian inter-laminar vertebral implant apparatus according to various embodiments.

FIG. 1B is a simplified top view of a mammalian inter-laminar vertebral implant apparatus according to various embodiments.

FIG. 1C is a simplified front view of a mammalian inter-laminar vertebral implant apparatus according to various embodiments.

FIG. 1D is a simplified back view of a mammalian inter-laminar vertebral implant apparatus according to various embodiments.

FIG. 1E is a simplified bottom view of a mammalian inter-laminar vertebral implant apparatus according to various embodiments.

FIG. 1F is a simplified cross-sectional top view of a mammalian inter-laminar vertebral implant apparatus across line A-A shown in FIG. 1C according to various embodiments.

FIG. 1G is a simplified isometric diagram of another mammalian inter-laminar vertebral implant apparatus according to various embodiments.

FIG. 2A is a simplified side diagram of a plurality of mammalian vertebrae that may be treated according to various embodiments.

FIG. 2B is a simplified rear diagram of a plurality of mammalian vertebrae that may be treated according to various embodiments.

FIG. 3A is a simplified, exploded side diagram of a plurality of mammalian vertebrae including laminar removed prior to an implant apparatus implantation according to various embodiments.

FIG. 3B is a simplified side diagram of a plurality of mammalian vertebrae including an implanted inter-laminar vertebral implant apparatus according to various embodiments.

FIG. 3C is a simplified rear diagram of a plurality of mammalian vertebrae including an implanted inter-laminar vertebral implant apparatus according to various embodiments.

FIG. 3D is a computer illustrated side diagram of a plurality of mammalian vertebrae including an implanted inter-laminar vertebral implant apparatus according to various embodiments.

FIG. 3E is a computer illustrated rear diagram of a plurality of mammalian vertebrae including an implanted inter-laminar vertebral implant apparatus according to various embodiments.

FIG. 4A is a simplified isometric diagram of another mammalian inter-laminar vertebral implant apparatus according to various embodiments.

FIG. 4B is a simplified top view of a mammalian inter-laminar vertebral implant apparatus according to various embodiments.

FIG. 4C is a simplified front view of a mammalian inter-laminar vertebral implant apparatus according to various embodiments.

FIG. 4D is a simplified back view of a mammalian inter-laminar vertebral implant apparatus according to various embodiments.

FIGS. 5A-5K are computer illustrated isometric diagrams of other mammalian inter-laminar vertebral implant apparatuses according to various embodiments.

FIG. 6 is a flow diagram illustrating mammalian inter-laminar vertebral implant apparatus implantation algorithms according to various embodiments.

### DETAILED DESCRIPTION

Spinal degeneration is aging process and may cause several clinical problems such as disc herniation or spinal stenosis. As spinal components degenerate, adjacent spinal ligaments and joints may become enlarged to attempt to maintain spinal stability. This degenerative process may reduce the spinal canal or neural foramen formed by spinal vertebrae. Such vertebrae space reduction is known as stenosis and may lead to patient discomfort and require intervention.

Patients with compromised vertebrae spacing may be treated non-surgically depending on the level of space reduction. Some patients, however may require surgical intervention or treatment. Such surgery may include removing spinal disc, bone, or ligaments to increase desired spacing to reduce or stop nerve tissue impingement including debriding related spinal vertebrae facet joints.

Such surgical procedures, however, while temporarily increasing desired spacing may cause spinal instability, which may cause further spinal component degeneration. To prevent or limit such spinal instability after such surgery, other procedures may be required to provide spinal stasis or stability. In addition, other techniques or procedures may be employed to provided desired spinal spacing while providing or maintaining spinal stability.

In addition, patient back pain may be caused by a variety of conditions, including intervertebral disc herniation, degenerative disc disease, facet degeneration, arthritis, noted spinal stenosis, spinal instability, and trauma to vertebrae, muscles, or ligaments. A spinal disc herniation may be treated by partial disc removal (discectomy). As noted, spinal stenosis may be surgically treated by various spinal bone removal techniques including laminectomy and facetectomy. In addition, adjacent vertebrae may be fused (no movement between the vertebrae) to address some spinal instability conditions, including degenerative disc disease, stenosis, scoliosis and spondylolisthesis.

Lumbar spinal fusion may be the most common spinal operation in the United States and South Korea to address some spinal instability conditions and undesired spacing between components. Lumbar spinal fusion procedures may employ interbody fusion cages, pedicle screws, interconnecting rods, and other components. Some spinal fusion procedures may be conducted via an open surgical procedure or a minimally invasive surgery (MIS). Open surgical procedures and MIS to deploy pedicle screws, rods, and related implants (usually in disc space) may be complex, requiring many specialized instruments and devices.

Alternatives to pedicle screw based spinal fusion have been developed. Such alternatives include non-fusion pedicle screw based stabilization and inter-spinous fusion systems. Non-fusion pedicle screw based stabilization procedures are still complex and have not had clinical success. Inter-spinous fusion system may employ an inter-spinous fusion device “IFD” and be employed with a disc spacer including Anterior Lumbar Interbody Fusion (ALIF), Posterior Lumbar Interbody Fusion (PLIF), and Transforaminal Lumbar Interbody Fusion (TLIF) spacers. Procedures to deploy an IFD are less complex than pedicle-rod implantations but have innate limitations including use for L5-S1 fusions.

More critically, deployed IFD may cause spinous process fractures due to the mechanical strain caused by such devices. Consequently, IFD based procedures have causes complications including spinous fractures, failure to improve patient pain, and a high rate of revision surgery. Further, a fusion employing an IFD may have biomechanical issues since the implanted IPD is located a long distance the spine's center of rotation.

The present invention includes an Inter-laminar vertebral implant apparatus (ILVIA) where the ILVIA is placed adjacent vertebrae bodies spinal canal, much closer to the spine's center of rotation. Such a placement is more stable than an IFD and less lightly to cause laminar fractures. Further, a ILVIA may be deployed in conjunction with a disc spacer (ALIF, TLIF, or PLIF). The ILVIA may be inserted as part of a decompression (spinal space increased) procedure.

One goal of spinal surgery may be to prevent adhesion where adhesion between bony structures may cause recurring pain thereafter. Implantation of an ILVIA of the present invention may act as a physical barrier in a decompressed region, preventing such adhesions. Spinal surgery may also cause radiculopathy, pain and muscular weakness. Over time after spinal surgery, affected spinal dural sac regions maybe be pushed or recede backwards due to gravity causing radiculopathy, pain and weakness. Implantation of an ILVIA of the present invention may present physical barrier the spinal dural sac and prevent is movement and associated radiculopathy, pain and muscular weakness.

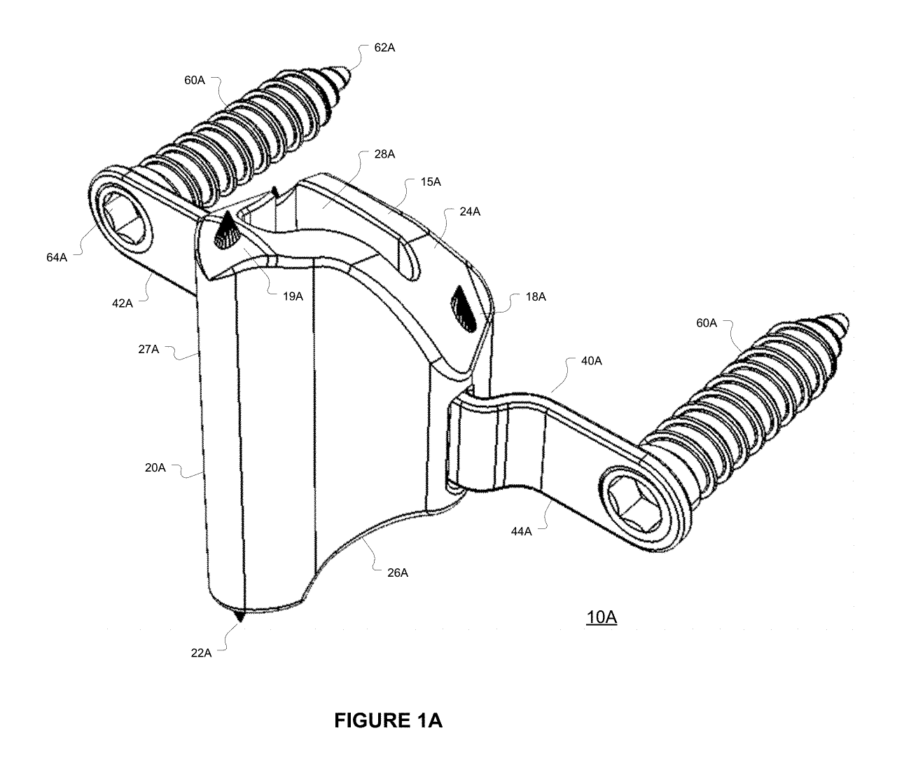

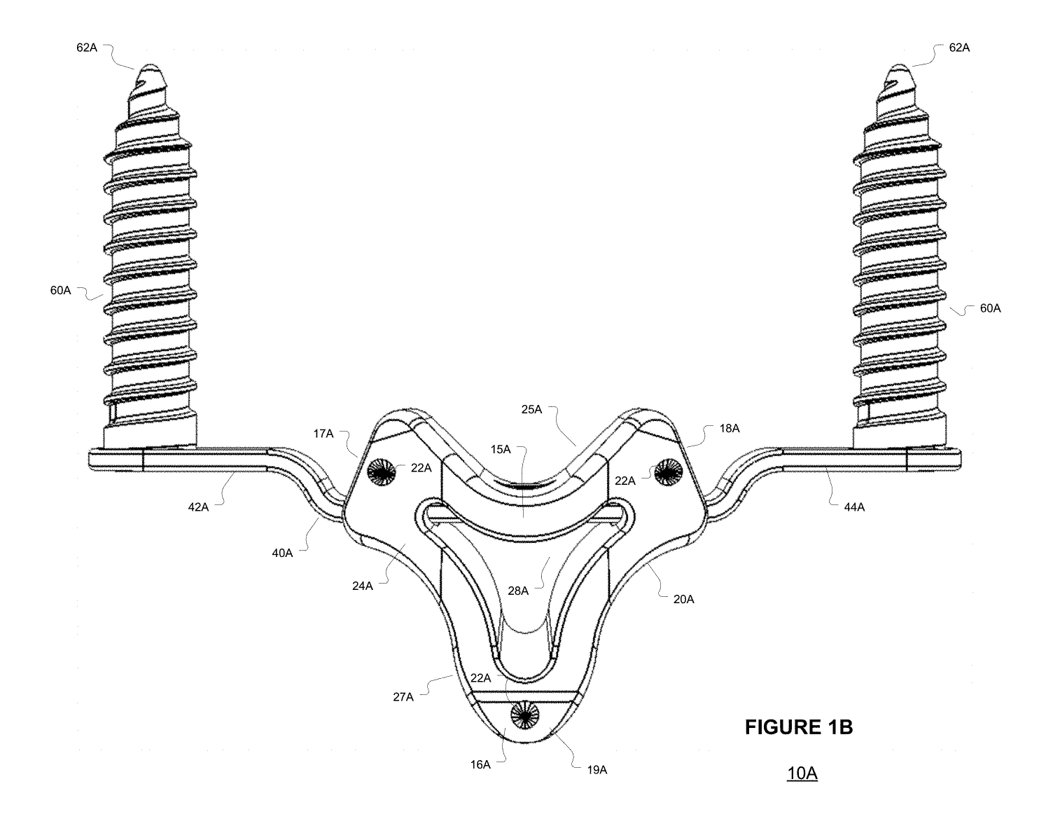

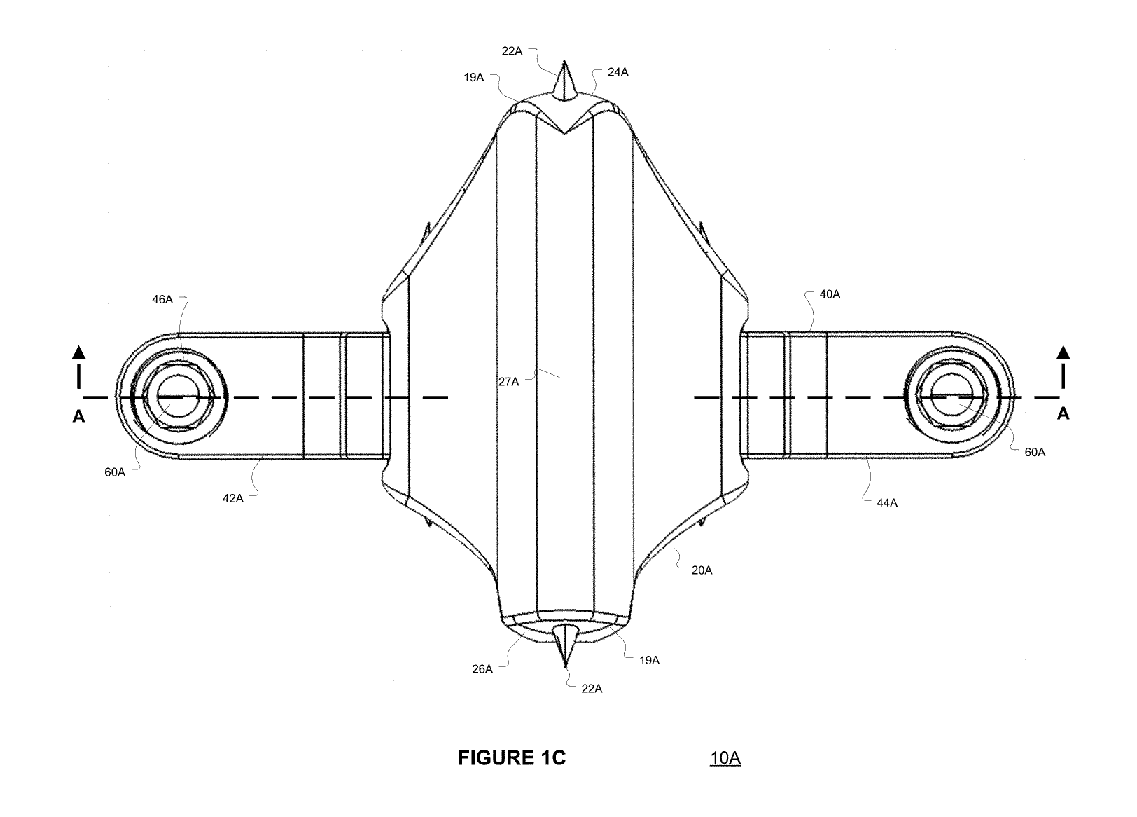

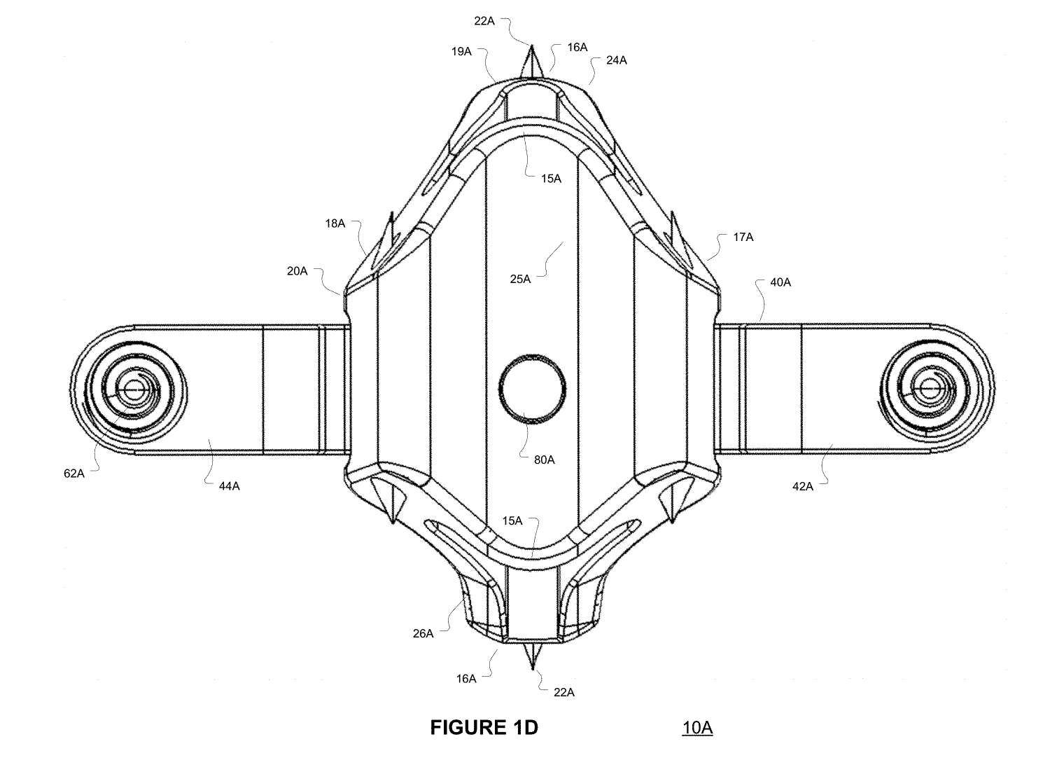

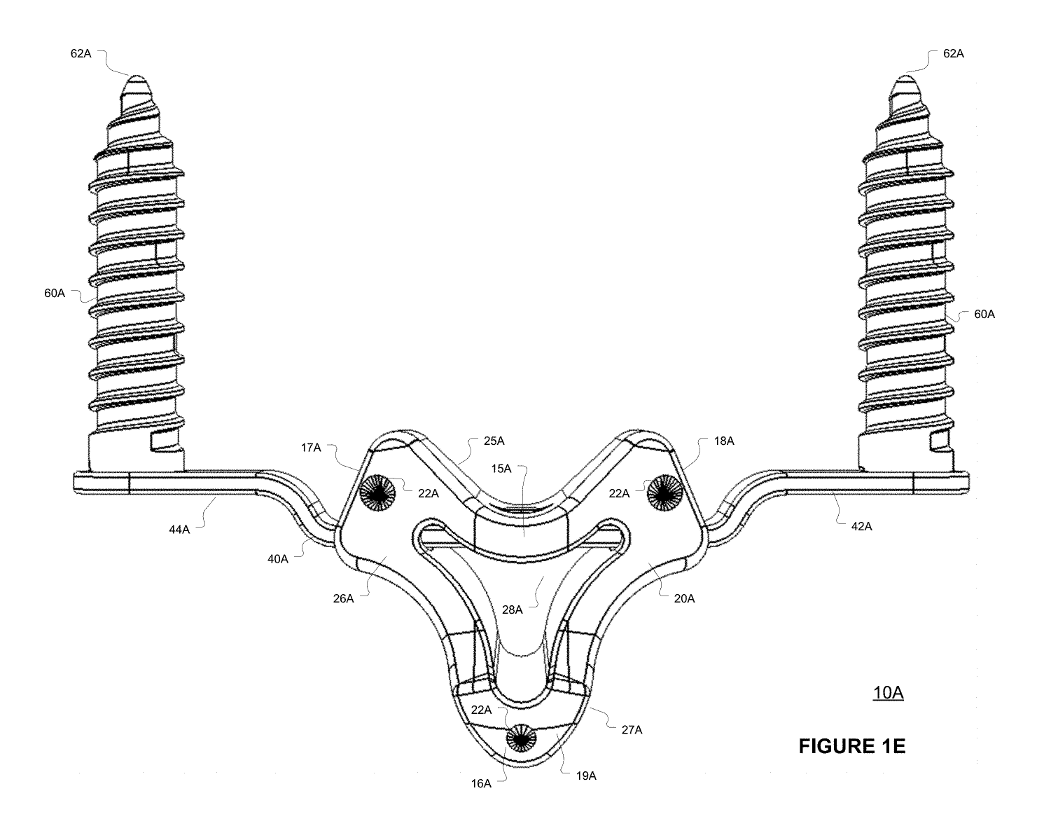

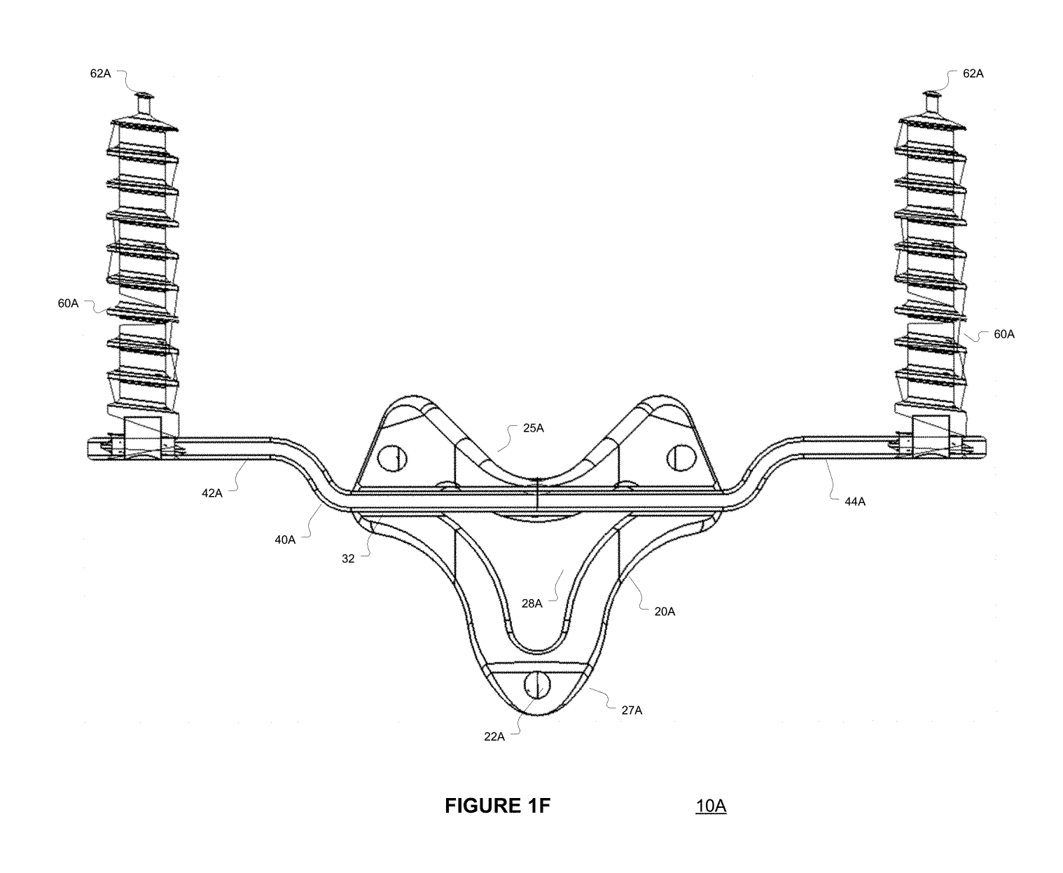

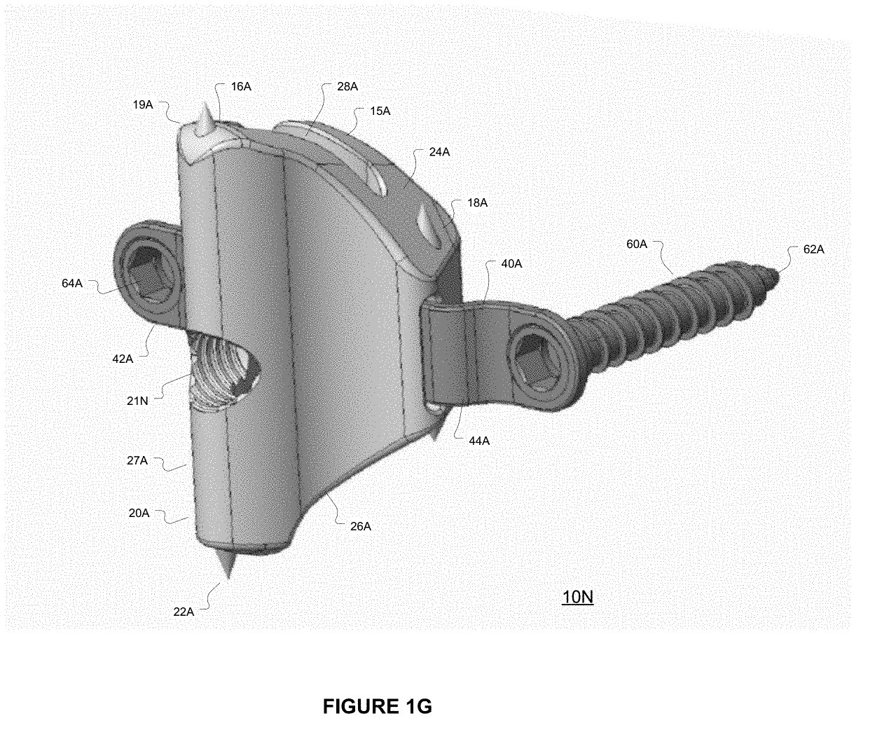

FIG. 1A is a simplified isometric diagram of a mammalian ILVIA 10A according to various embodiments. FIG. 1B is a simplified top view, FIG. 1C is a simplified front view, FIG. 1D is a simplified back view, and FIG. 1E is a simplified bottom view of the mammalian ILVIA 10A according to various embodiments. FIG. 1F is a simplified cross-sectional top view of the mammalian ILVIA across line A-A shown in FIG. 1C according to various embodiments. As shown in FIGS. 1A-1F, the ILVIA 10A includes a cage 20A, plate module 40A, and bony fixation elements 60A.

The cage 20A includes a top 24A with one or more protrusions 22A, a bottom 26A with one or more protrusions 22A, a fenestration 28A extending from the top 24A and bottom 26A. As shown in FIGS. 1A-1F, the cage 20A has a concave front section/side 25A and a convex rear section/side 27A. The cage 20A is configured to be placed between two, adjacent vertebrae lamina and substantially adjacent the spinal canal formed by the vertebrae lamina. The cages 20A concave front section/side 25A may be configured to prevent reduction or impingement of the spinal canal formed by adjacent vertebrae. The fenestration 28A extending from the cage 20A top 24A to bottom 26A may be filled with bony fusion material including autogenous bone, artificial bony fusion promoting materials such as demineralized bone matrix (DBM) and bone morphogenetic protein (BMP), or combinations of both. In an embodiment, the concave front section/side 25A length from top 24A to bottom 26A may be less the convex rear section/side 27A length from top 24A to bottom 26A.

As shown in FIGS. 1A-1F, a cage 20A may include a slot 32A where an elongate plate 40A may be employed. In an embodiment, a pin 80A may be inserted into the plate 40A via a cage 20A concave front section/side 25A to secure the plate 40A within the cage 20A plate slot 32A. A plate 40A in an embodiment may have an elongated length extending between a first arm 42A and a second arm 44A. Each arm 42A, 44A may include a bony fixation element (bone screw in an embodiment) interface or opening 46A. The screw interface or opening 46A may be sized to enable a screw 60A distal end 62A to pass there-through but capture the screw head 64A. A screw 60A head 64A may include a tool interface, hexagonal or other compatible shapes. FIG. 1N is a simplified isometric diagram of a mammalian ILVIA 10N according to various embodiments including a threaded recess 21N in cage 20A convex rear section/side 27A. The threaded recess 21N may be couplable to a tool to aid a surgeon to place a ILVIA 10A-N into a desired location between two bony structures.

As shown in the figures, cage 10A-M each has three arms or legs including a front side left arm/leg 17A-M, a front side right arm/leg 18A-M, and a rear side central arm/leg 19A-M. Each left arm/leg 17A-M, right arm/leg 18A-M, and a rear side central arm/leg 19A-M may include an upward protrusion 22A-22B on the respective cage 20A-M top side or portion 24A-M and bottom side or portion 26A-M. As also shown in figures, the left arm/leg 17A-M, right arm/leg 18A-M, and a rear side central arm/leg 19A-M may form vertices of a triangle with sides having a substantially constant width between the legs/arms forming a central fenestration 28A-M extending from the cage 20A-M top side/portion 24A-M to bottom side/bottom 26A-M.

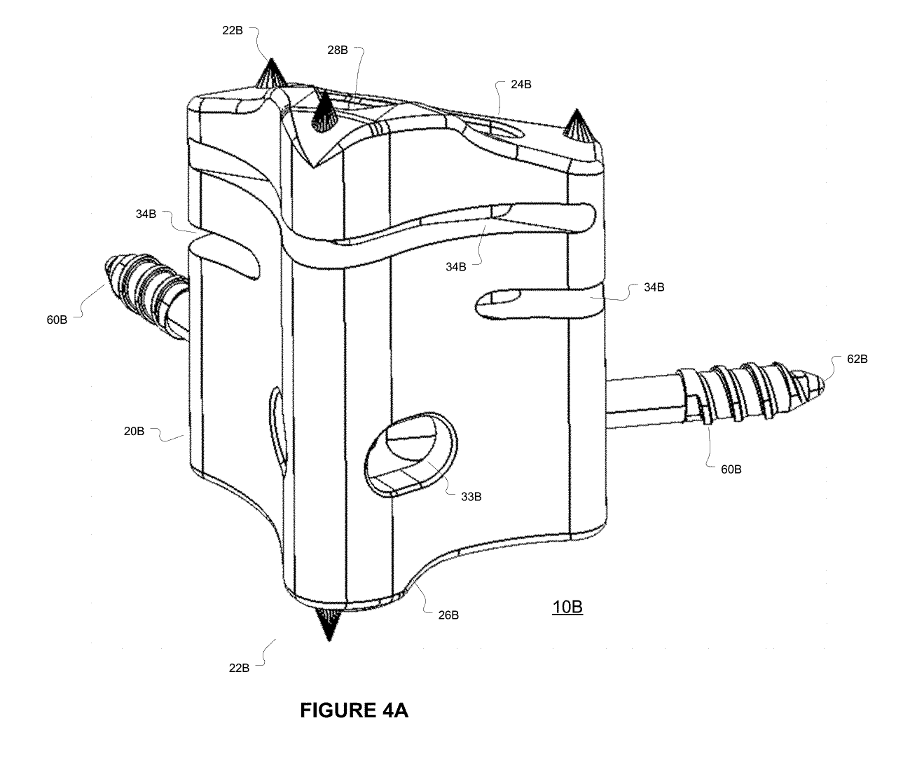

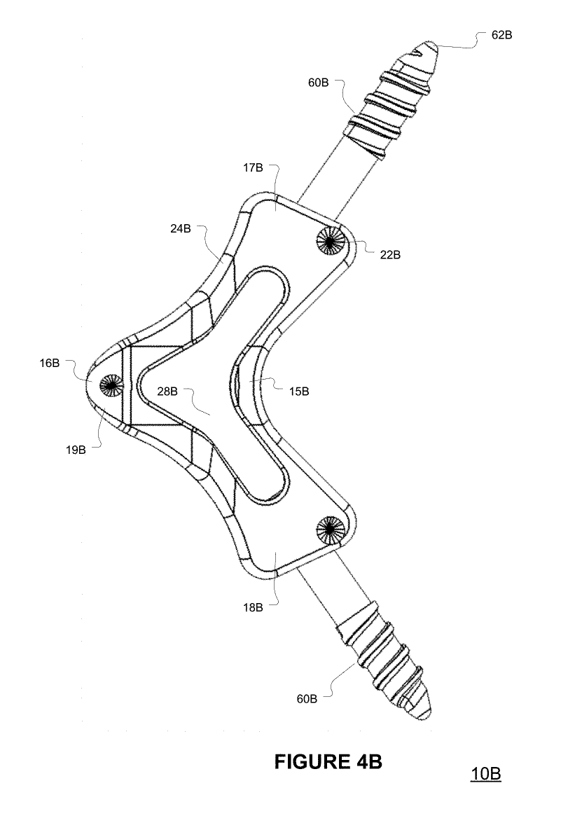

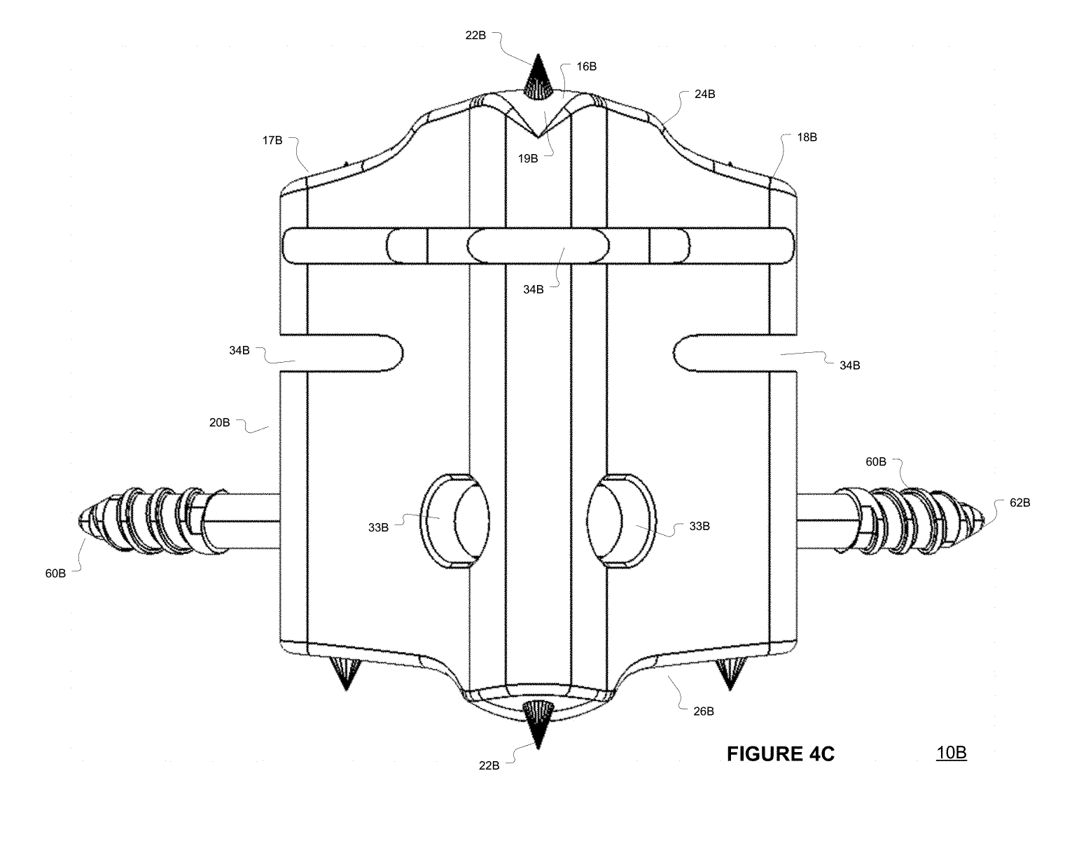

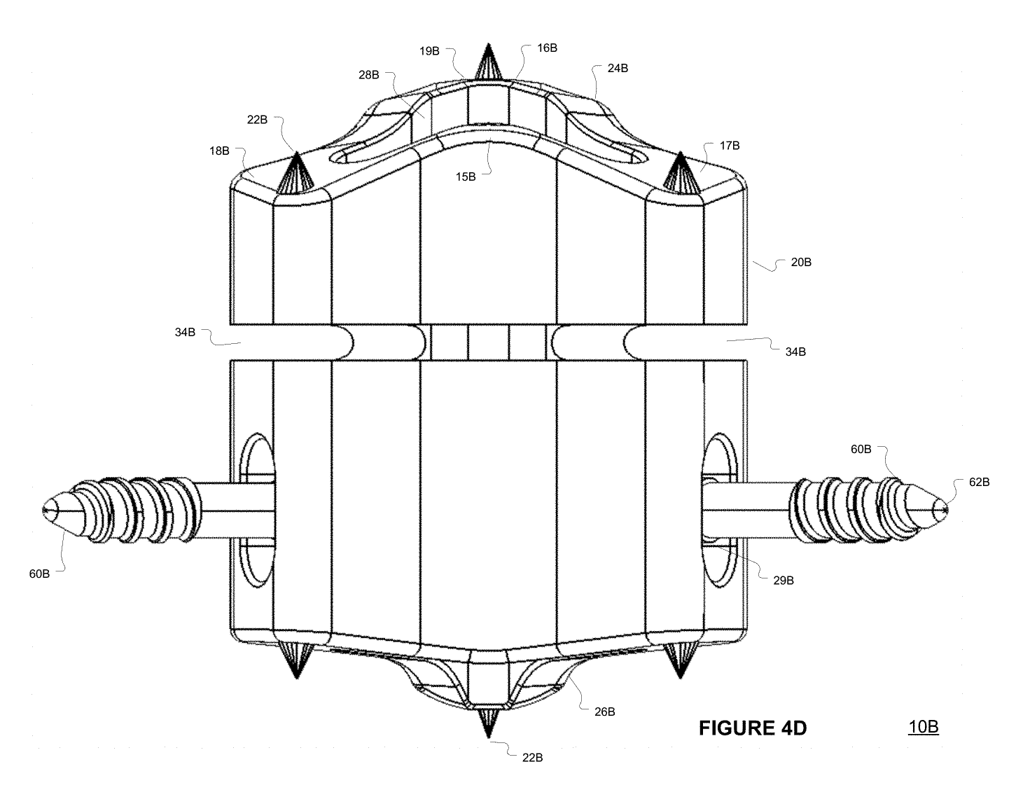

As shown in FIGS. 1A-1N and 4A-4D the cages 10A and 10B top sides 24A, 24B and bottom sides 26A, 26B may include a front peak 15A, 15B and a rear peak 16A, 16B. The front peak 15A, 15B may extend between the front arms 17A, 17B and 18A, 18B and slope down equally to both arms 17A, 17B and 18A, 18B. The rear peak 16A, 16B may be located in the rear central arm 19A, 19B and slope down from the cage 20A, 20B rear to front equally to both arms 17A, 17B and 18A, 18B. As also shown in FIGS. 1A-1N and 4A-4D, the front peak 15A, 15B may be lower in height than the rear peak 16A, 16B. As further shown in FIGS. 1A-1N and 4A-4D, the left arm/leg 17A-B, right arm/leg 18A-B, and a rear side central arm/leg 19A-B may form vertices of a triangle having inwardly curved sides having a substantially constant width between the legs/arms forming a central fenestration 28A-B extending from the cage 20A-B top side/portion 24A-B to bottom side/bottom 26A-B.

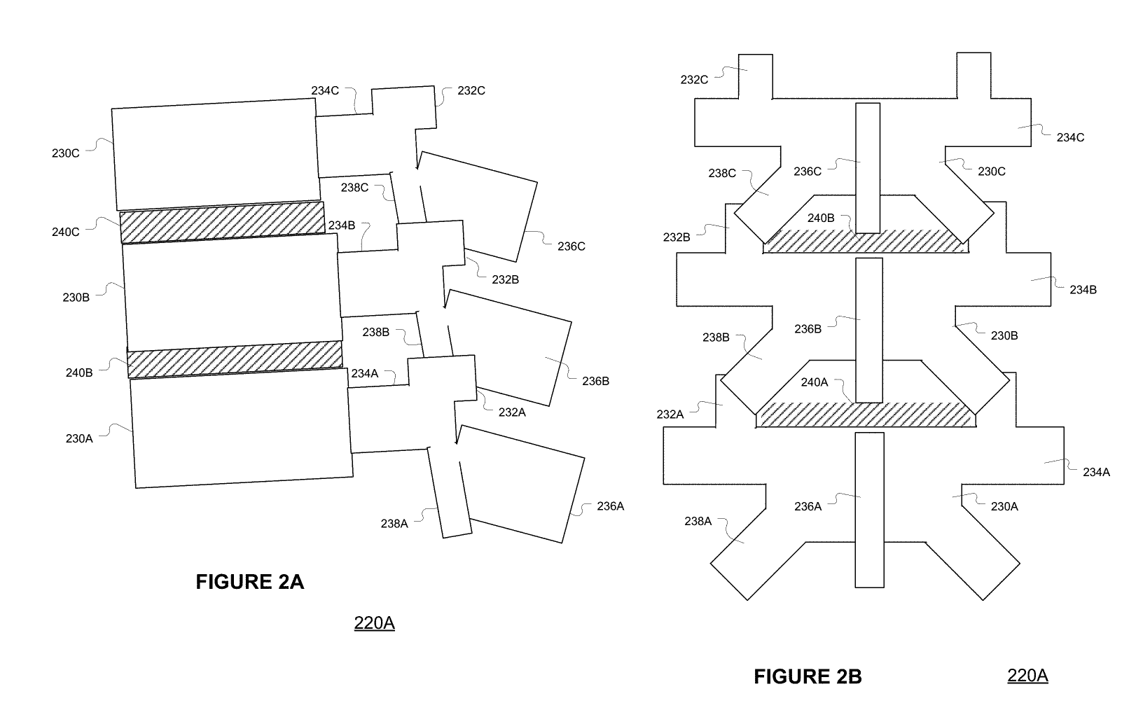

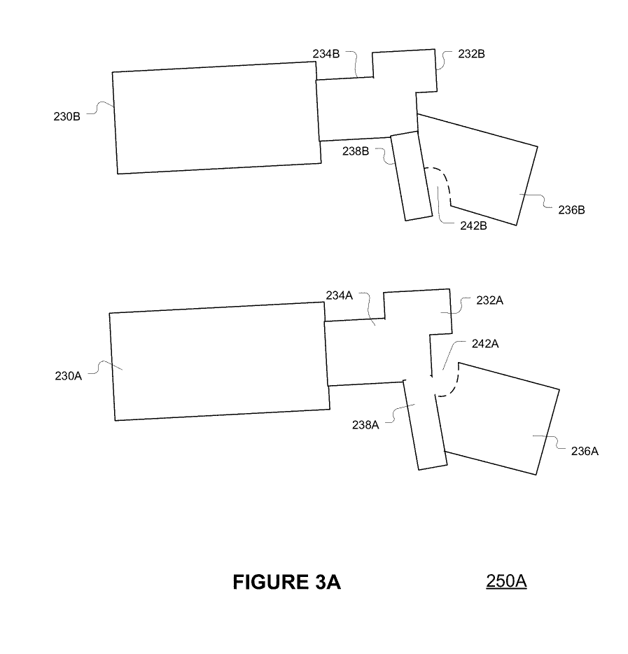

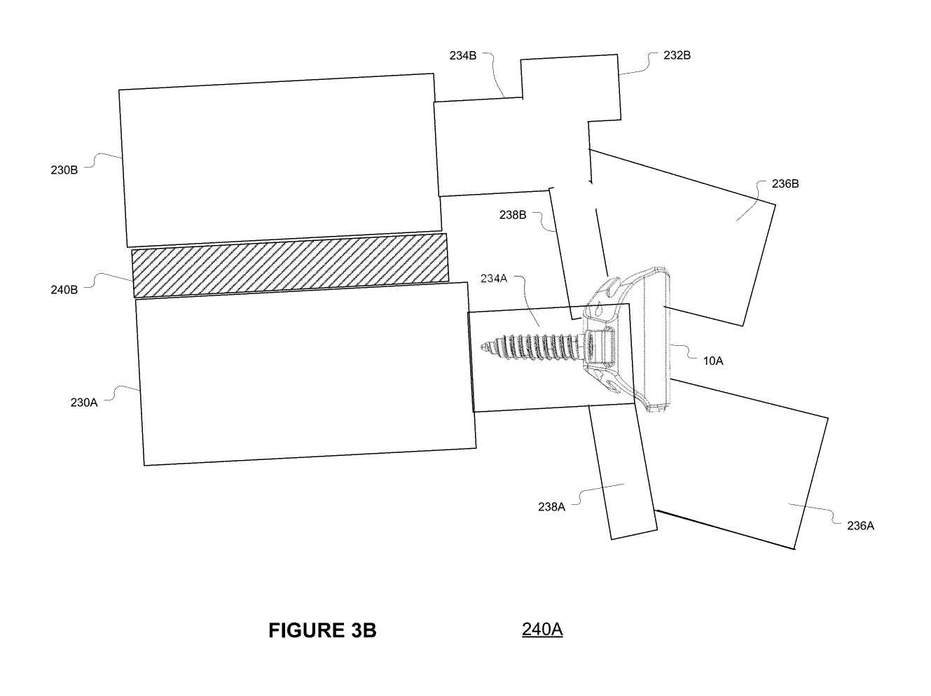

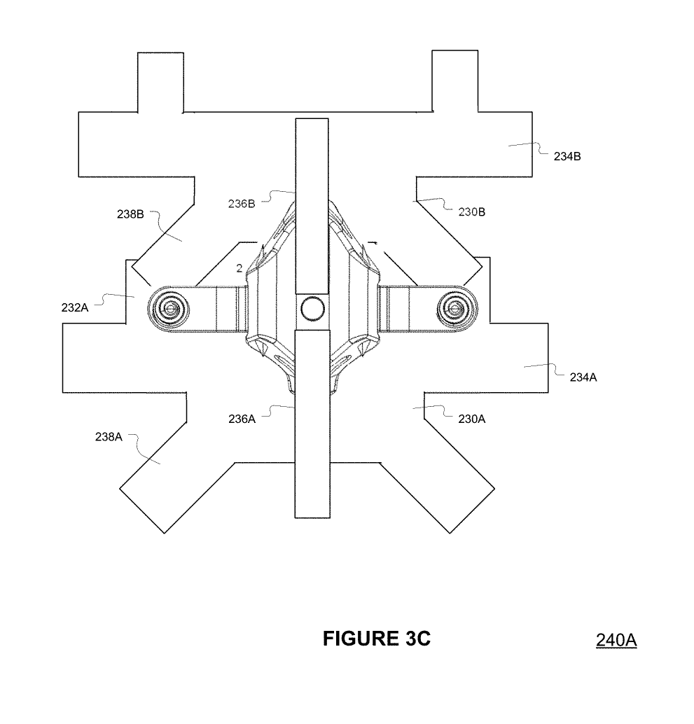

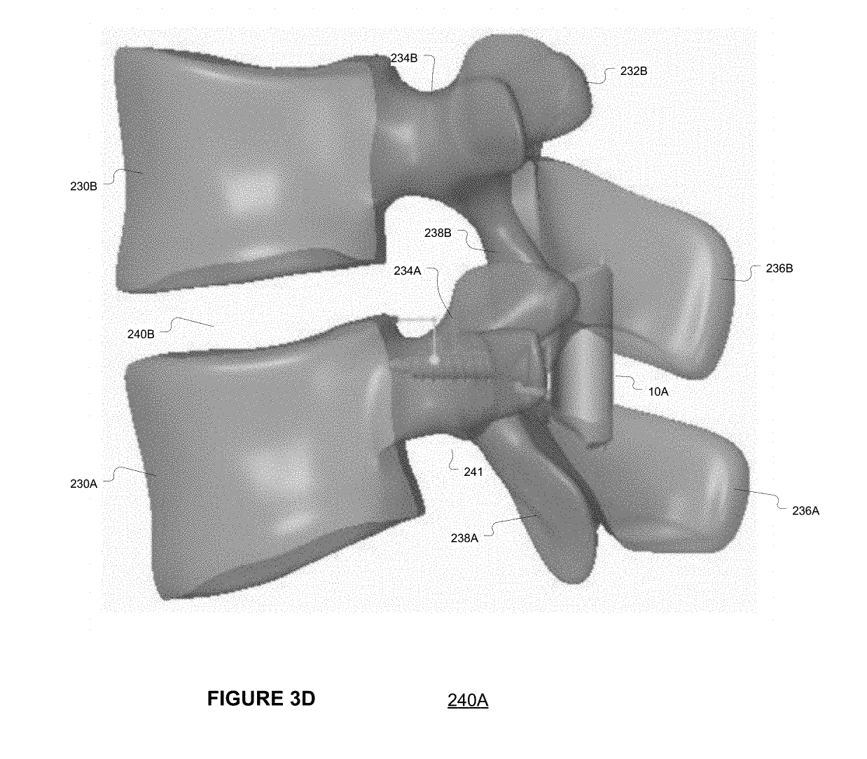

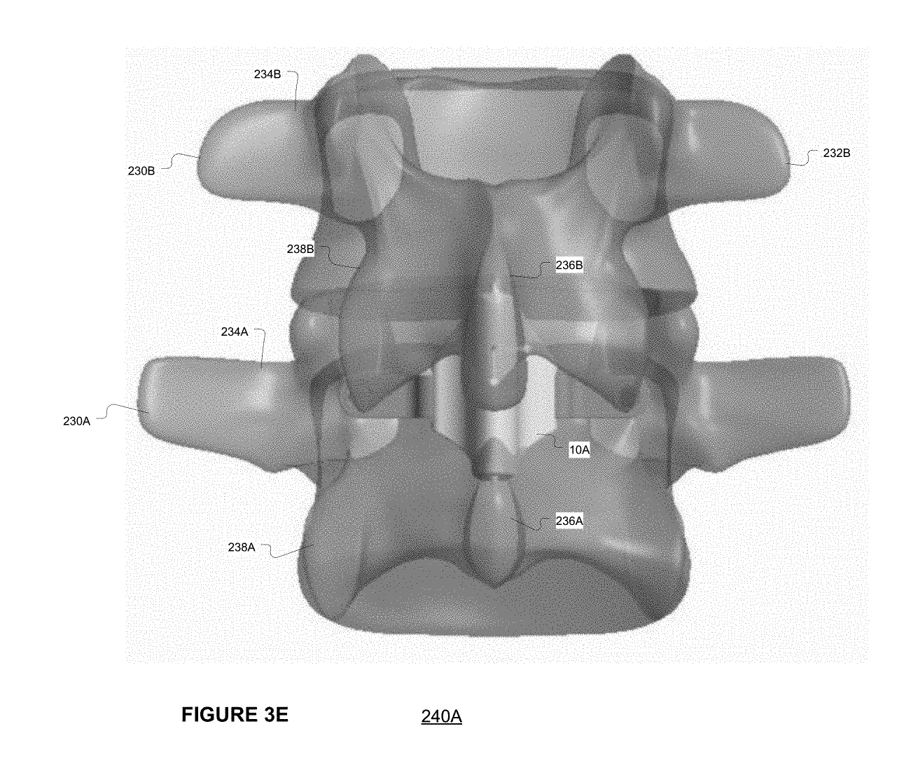

In an embodiment the plate arms 42A, 42B are sized in length to enable the screws 60A coupled therein to engage pedicles of a lower vertebrae 230A of a vertebrae pair 230B, 230A where the ILVIA 10A is deployed or implanted. The protrusions 22A on the cage 20A top 24A and bottom 26A may be sized to and shaped to engage vertebrae lamina in an embodiment. The combination of the cage 20A size, shape, protrusions 22A, and plate 40A with screws 60A may enable a surgeon to securely couple the ILVIA between two vertebrae 230A, 230B as shown in FIGS. 3B-3E. FIG. 2A is a simplified side diagram and FIG. 2B is a simplified rear diagram of a plurality of mammalian vertebrae 230C, 230B, and 230A that may be treated according to various embodiments. As shown in FIGS. 2A and 2B, a vertebrae 230A-C may include pedicles 234A-C coupled to pedicle processes 232A-C, forming lamina 238A-C between the pedicles and coupled to the spinous processes 236A-C.

An ILVIA 10A of the present invention is configured sized to be placed into the lamina 238A-C and dorsal process 236A-C region immediately adjacent the lamina 238A-C. FIG. 3A is a simplified, exploded side diagram of a plurality of mammalian vertebrae 230B, 230A including lamina 242A, 242B removed prior to an ILVIA 10A implantation according to various embodiments. FIG. 6 is a flow diagram illustrating mammalian an ILVIA 10A implantation algorithm 300 according to various embodiments. In an embodiment, a surgeon may want to stabilize or fuse two, adjacent vertebrae 230B, 230A as shown in FIGS. 2A-3E. Via an open or MIS procedure, a surgeon may expose a space between adjacent vertebrae 230B, 230A where an ILVIA 10A is to be implanted (activity 302).

A surgeon may remove lamina in the upper vertebrae 230B of the pair 230B, 230A including in the lamina 238B and adjacent dorsal process 236B (shown partially as 242B in FIG. 3A, 250A) to provide room for the ILVIA 10A cage 20A upper section 24A. A surgeon may also remove lamina in the lower vertebrae 230A of the pair 230B, 230A including in the lamina 238A and adjacent dorsal process 236A (shown partially as 242A in FIG. 3A, 250A) to provide room for the ILVIA 10A cage 20A lower section 26A. (activity 304). A surgeon may then size the formed opening and choose an ILVIA 10A-M (shown in FIGS. 1A-1F, 4A-4D, and 5A-5K) where the different ILVIA 10A-M may have different sizes and configurations based on the formed opening and the vertebrae 230B, 230A to be stabilized or fused (vertebra maybe lumbar, thoracic, or cervical vertebrae in an embodiment). (activity 308). The selected ILVIA 10A-10M may be inserted into lamina opening (activity 310). The vertebrae 230B, 230A may be distracted prior to ILVIA insertion to ease such insertion in an embodiment.

Then, a surgeon may employ one or more bony fixation elements 60A in horizontal plates 40A, C, D, vertical plates 50C-50M, or directly via cage (20B) to secure the ILVIA 10A-M to vertebrae 230B, 230A (activity 312).

FIG. 3B is a simplified side diagram and FIG. 3C is a simplified rear diagram of a mammalian vertebrae 230B, 230A including an implanted ILVIA 10A according to various embodiments. FIG. 3D is a computer illustrated side diagram and FIG. 3E is a computer illustrated rear diagram of mammalian vertebrae 230B, 230A including an implanted ILVIA 10A according to various embodiments. As shown in FIGS. 3B-E, the ILVIA 10A is placed near the spinal canal 241 at the start of dorsal processes 236A, 236B. The plate 40A arms 42A, 44A extend horizontally outwardly from the cage 20A with a length that enables screws 60A to enable the pedicles 234A of the lower vertebrae 230A of the pair to be stabilized or fused.

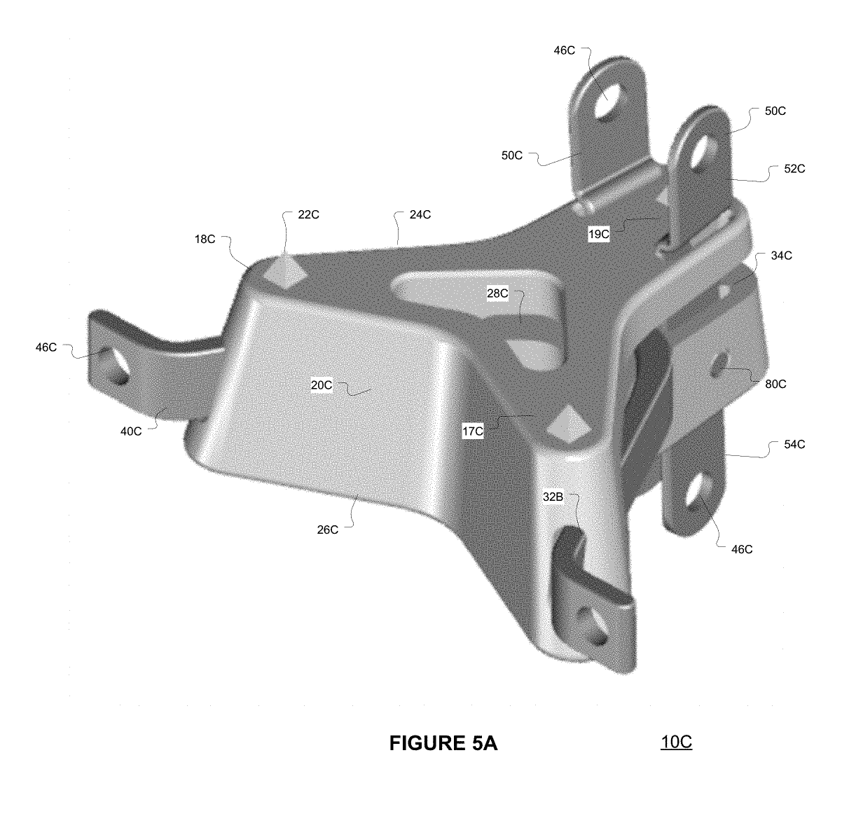

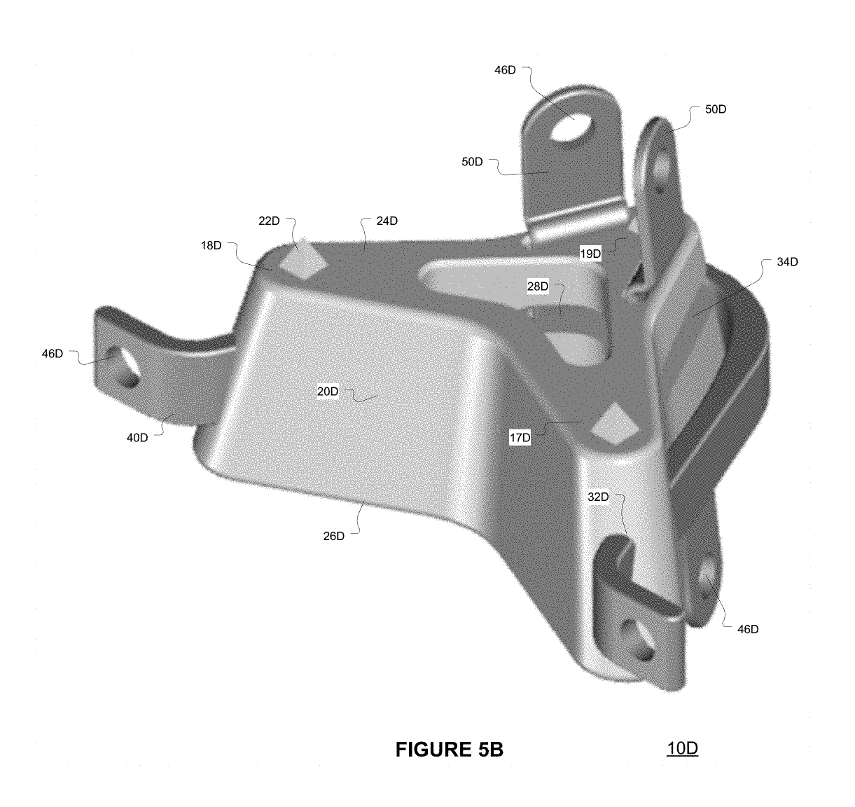

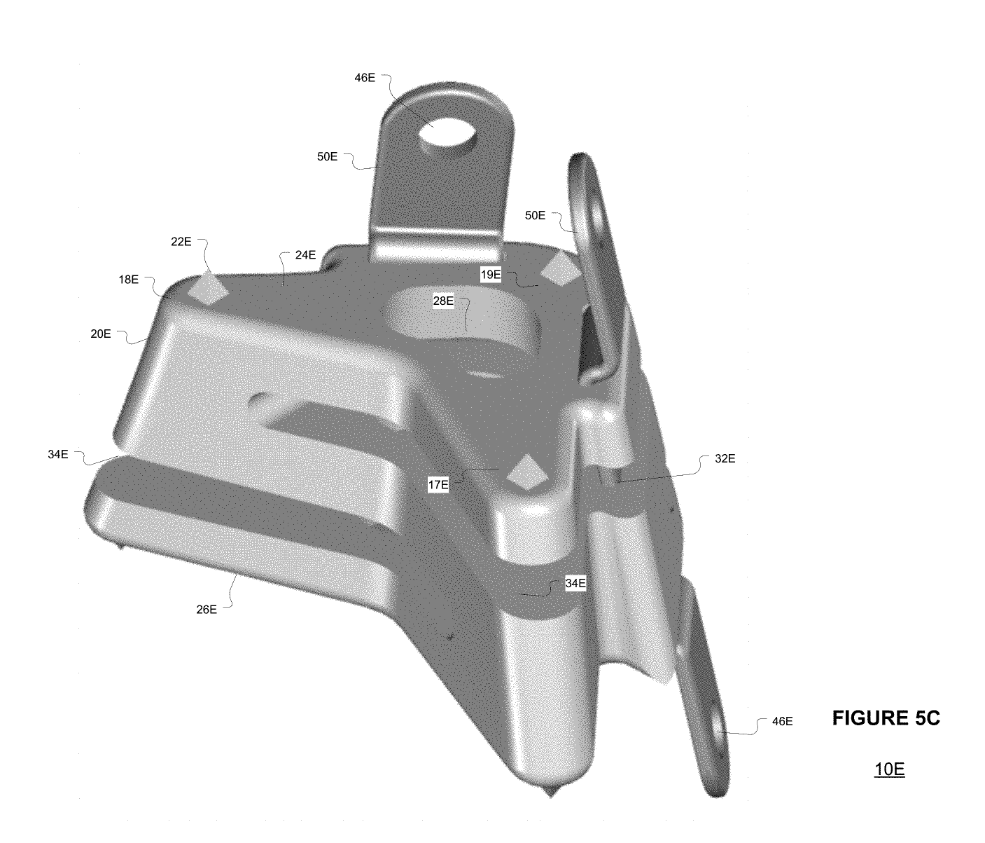

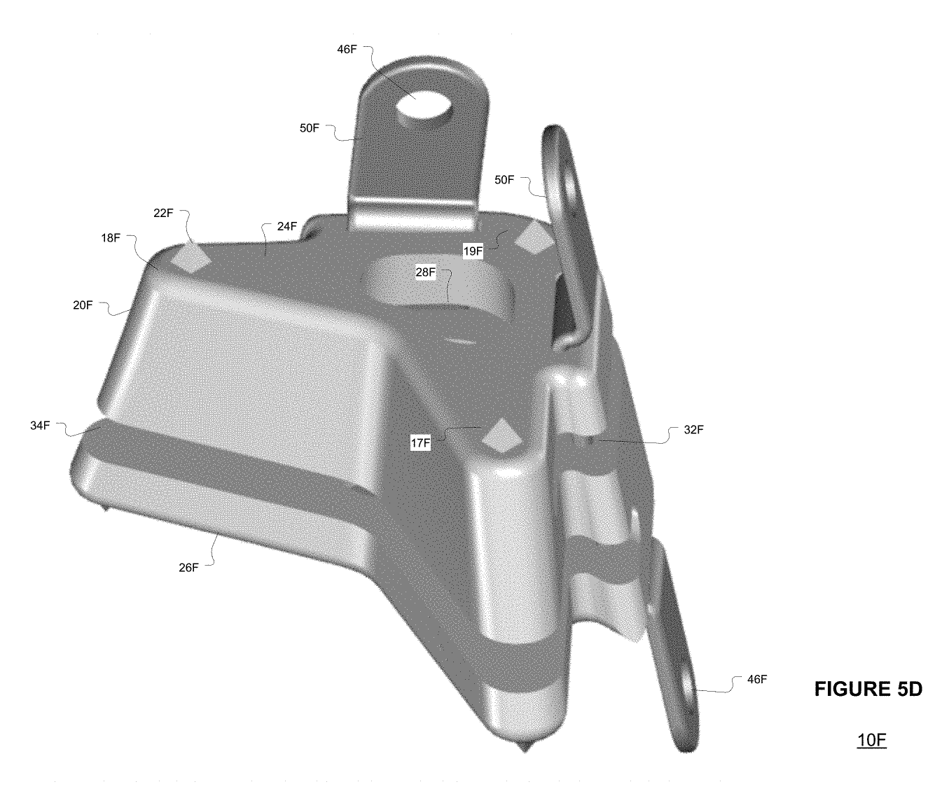

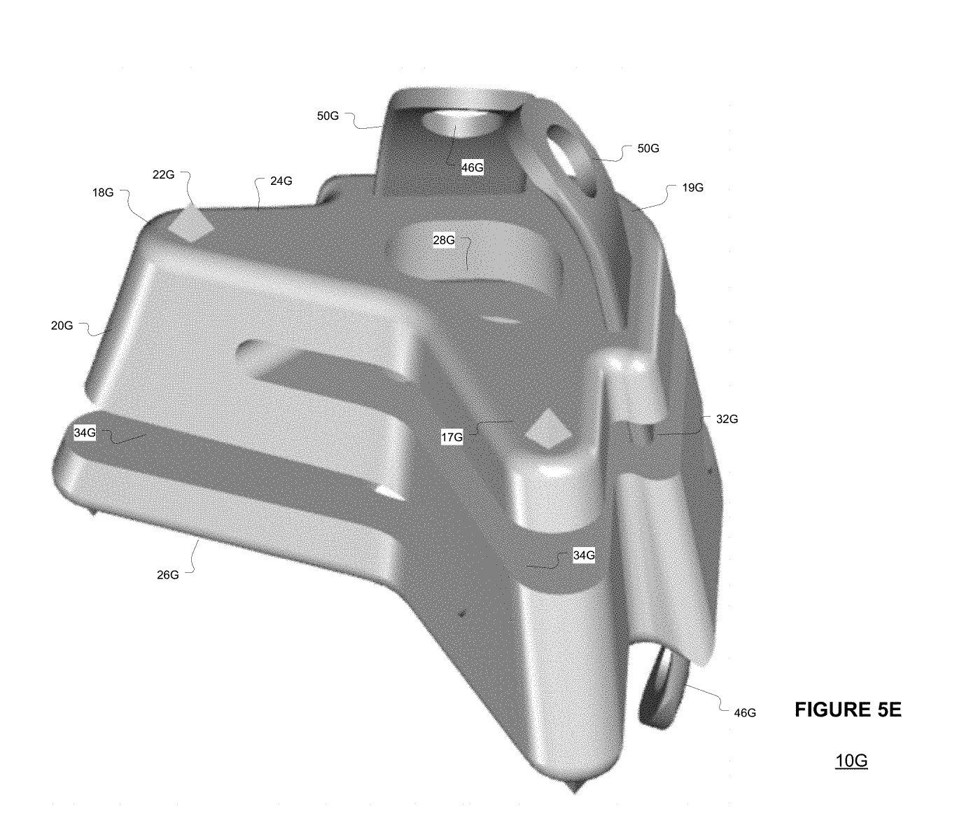

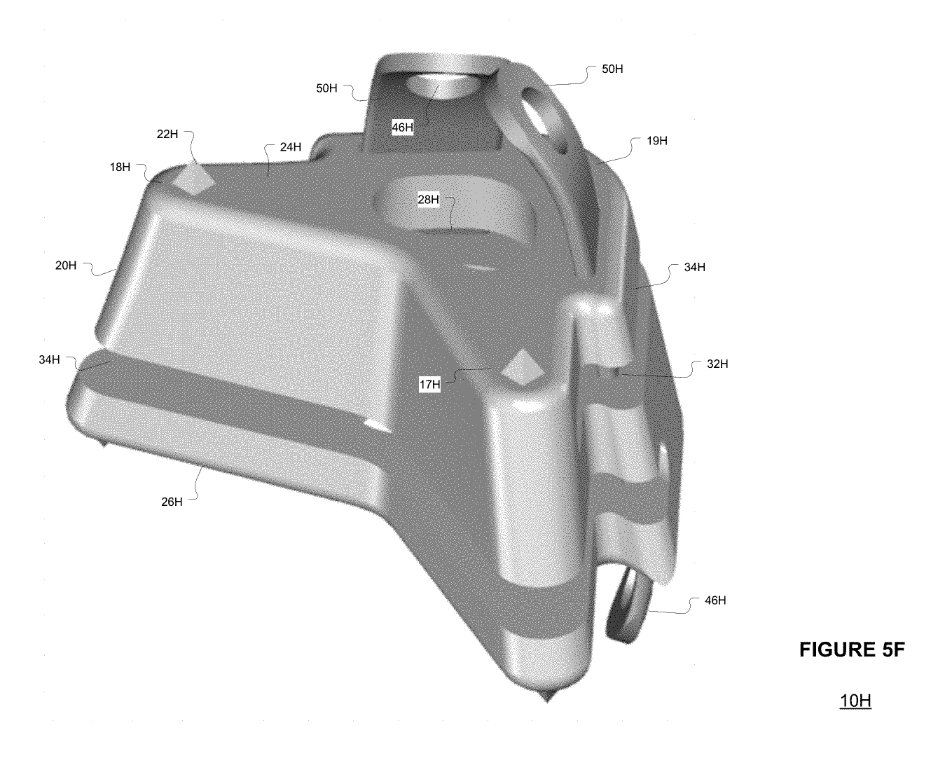

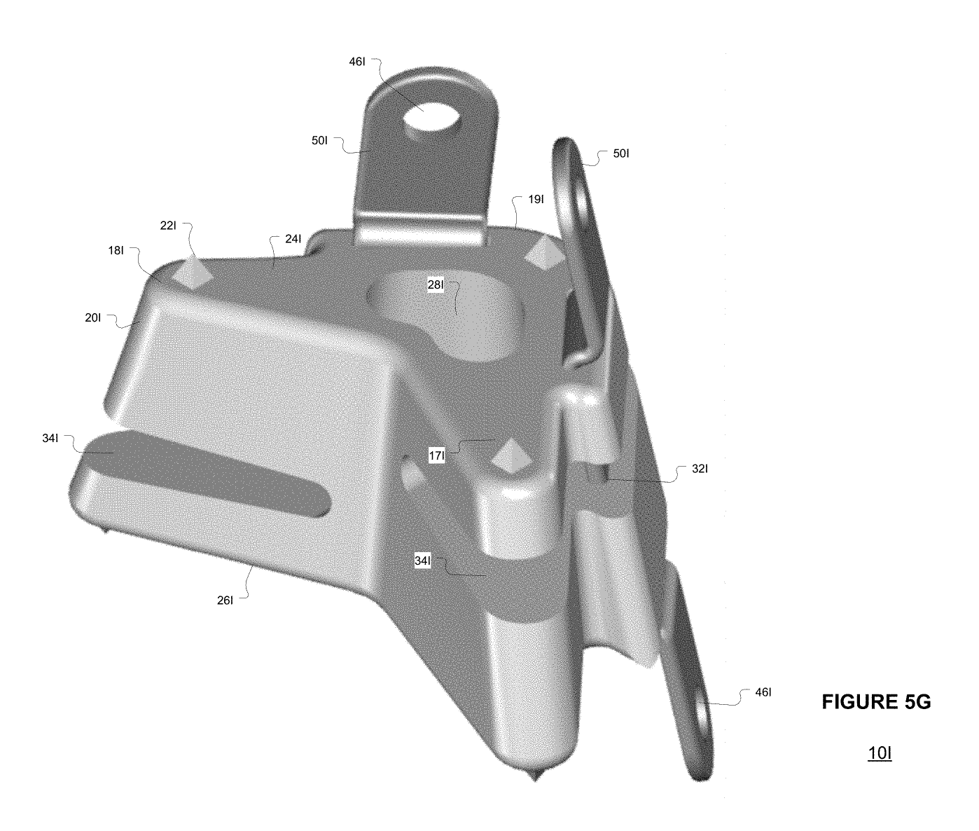

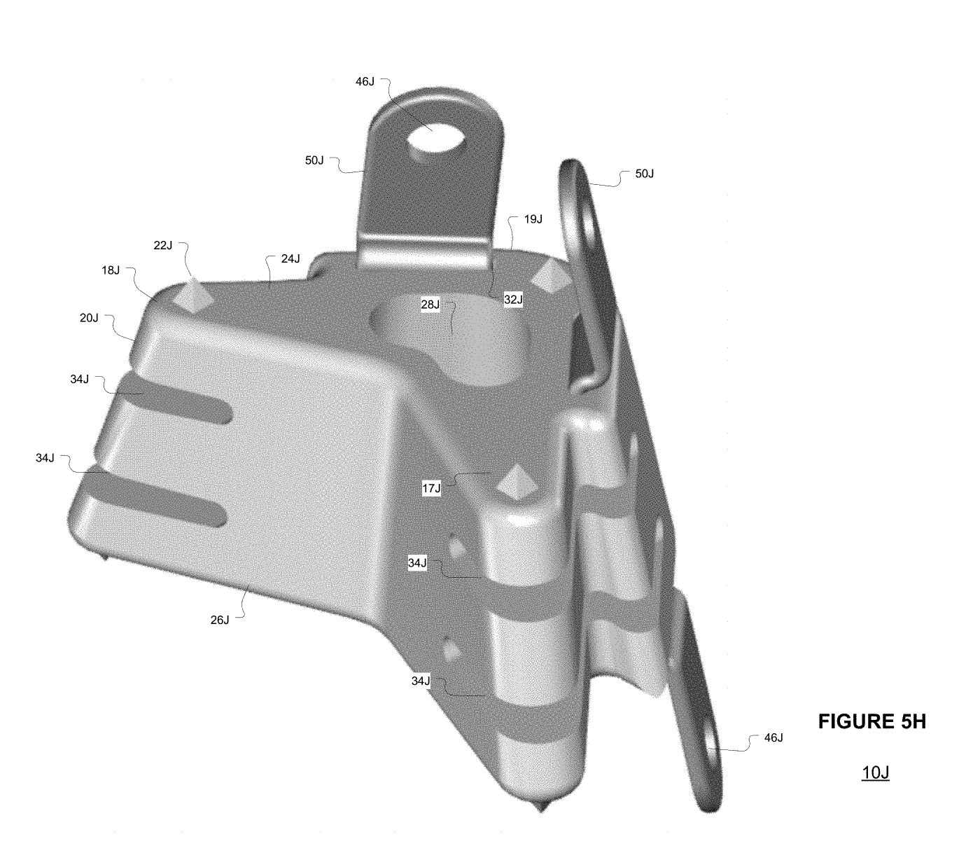

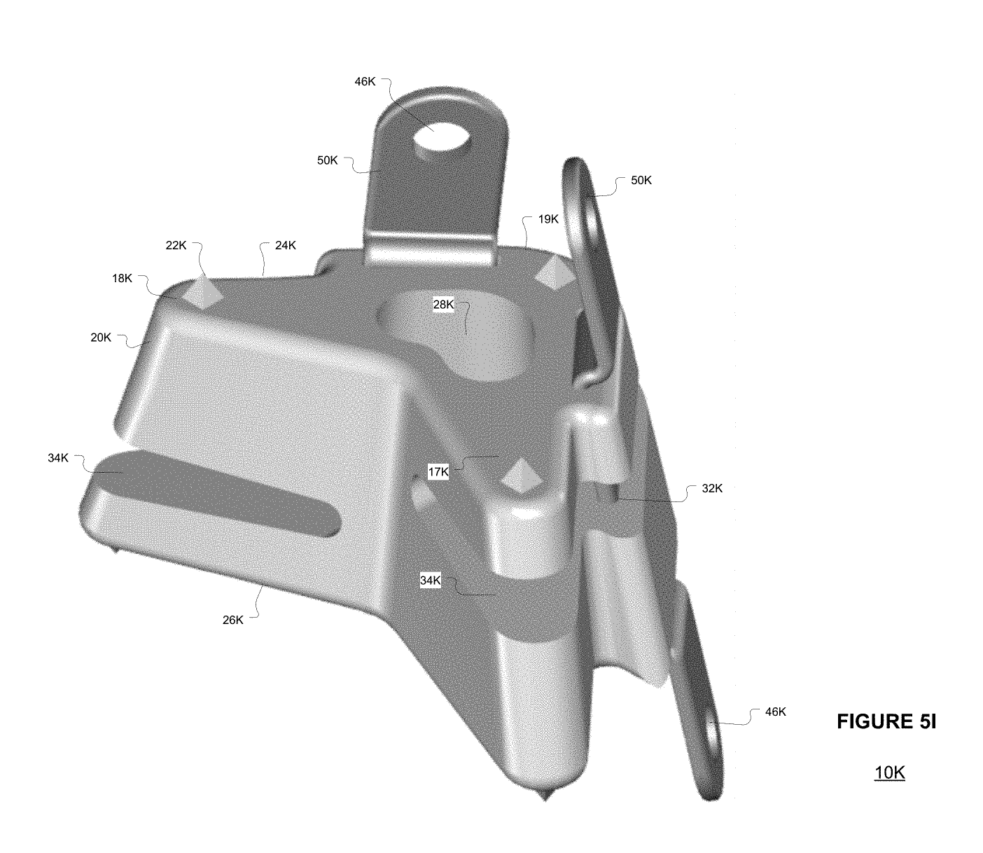

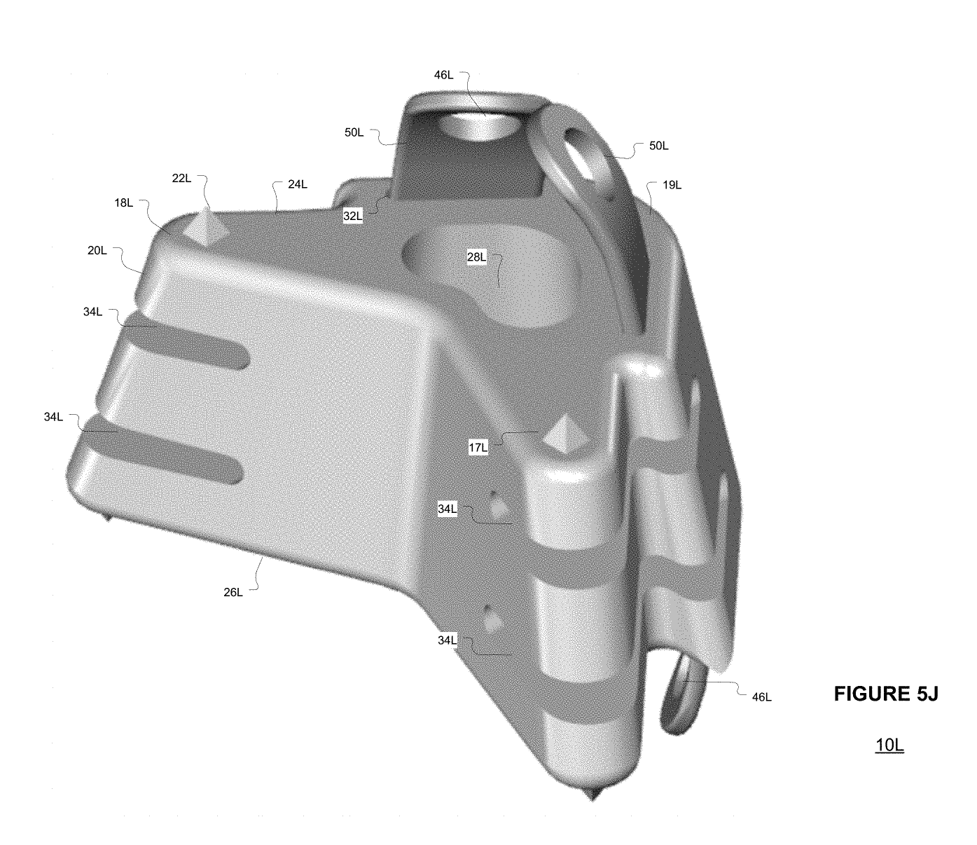

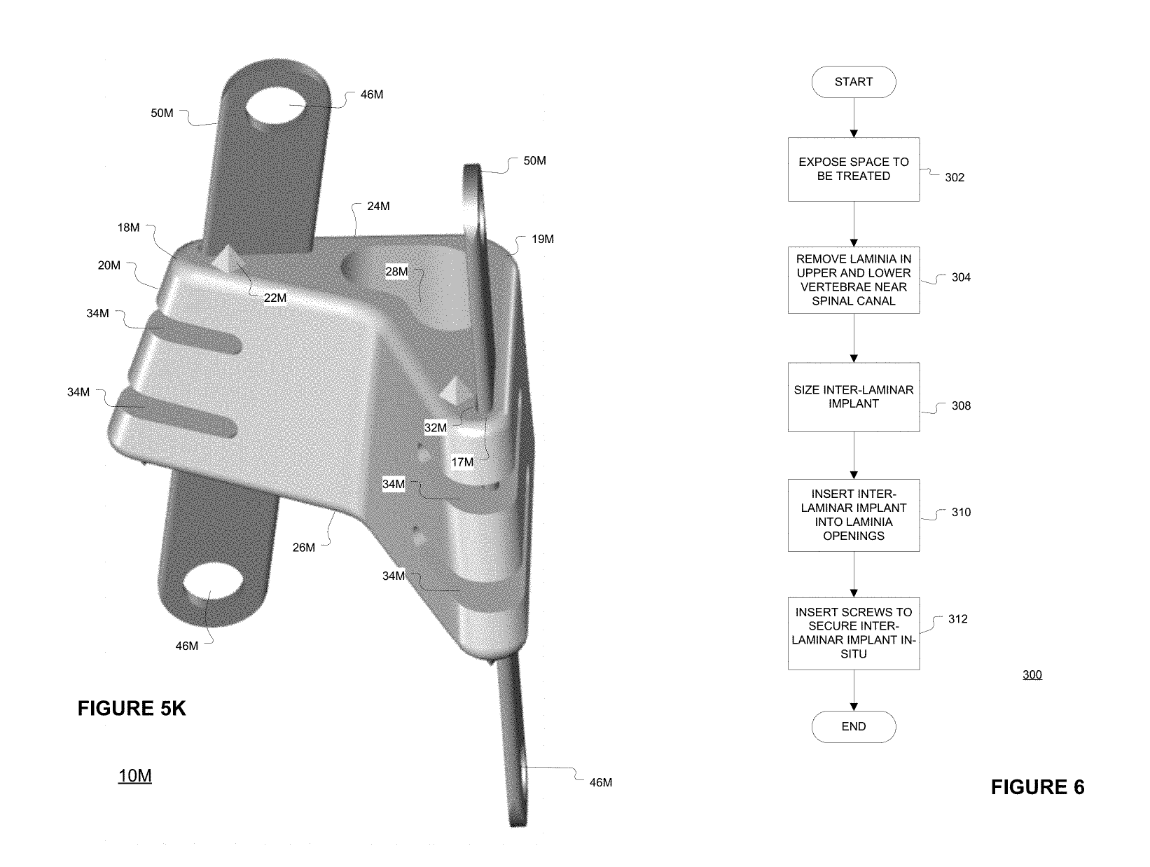

As noted, an ILVIA 10A-10M is sized and configured for implantation and/or affixation between the laminar 238A-C of adjacent superior and inferior vertebrae pairs 230A-C. As shown in FIGS. 5A-5K, ILVIA 10C-M may include vertical or vertically curved plates 50C-M. Such plates 50C-M may include an upper, cranial arm 52C, configured to contact an inferior surface of a spinous process 236B of a superior vertebral body (230B in FIGS. 2A-3E) and a caudal lower arm 54C for contacting a superior surface of the spinous process 236A of the inferior vertebral body (230A in FIGS. 2A-3E). In an embodiment, the cranial, upper arm 52C may be moveable with respect to the caudal, lower arm 54C so that an overall height of the spacer member is adjustable. The cranial and caudal arms 52C, 54C each may include one of first and second lateral projections extending therefrom or first and second lateral bores 46C formed therein.

As shown in FIGS. 5A to 5K, other ILVIA 20C-M may include vertical plates 50C-M that may be coupled to either lamina 238B, 238A of adjacent vertebrae 230B, 230A or their respective dorsal processes 236B, 236A. ILVIA 10C and 10D may include both horizontal plates 40C, 40D and vertical plates 50C, 50D. ILVIA 10B to 10M may include one or more slits 34B to 34M.

The slits may partial on edges or extend along a cage 20B-20M convex rear section/side 27B-27M or concave front section/side 25B-25M (such as shown FIGS. 10F and 10G. The slits 34B-34M may be sized or shaped to enable such flexion of the cage 20B-M to reduce or prevent adjacent level failures of vertebrae adjacent the stabilized vertebrae 230A, 230B. The cage 20A-M, plates 40A, 40C, 40D, and 50C-M, and bony fixation elements 60A may be formed out of any biocompatible and resilient material including polymers, ceramics, metals, alloys, or combinations thereof.

As noted a ILVIA may not include a plate where the bony fixation elements 60A may be coupled to vertebrae 230A, 230B via a cage 10B directly. FIG. 4A is a simplified isometric diagram, FIG. 4B is a simplified top view, FIG. 4C is a simplified front view, and FIG. 4D is a simplified back view of a mammalian ILVIA 20B that does not employ a plate to couple the ILVIA via a bony fixation element 60B to a vertebrae 230A-C when deployed according to various embodiments. As shown in FIGS. 4A to 4D, the cage 20B may include openings 33B that enable insertion of screws 60B into screw opening 29B. The openings 33B are also sized to enable a tool to operatively engage a screw 60B for deployment in or removal from a vertebrae 230A-C.

The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus the following claims are hereby incorporated into the Detailed Description, with each claim standing on its own as a separate embodiment.

What is claimed is:

1. A surgical implant for implantation between two adjacent bony structures, the implant including:
a cage including an elongated front left leg extending a first length from a top portion to a bottom portion, a front elongated right leg extending the first length from a top portion to a bottom portion, and an elongated rear leg extending a length from a top portion to a bottom portion, wherein the left leg, right leg, and rear leg form an appropriate triangle when viewed from the respective top portions, an inwardly curved front side extending from the front left leg to the right leg along the first length forming a concave section between the left leg and right leg, a left side extending from the left leg to the rear leg, a right side extending from the right leg to the rear leg, the front side, left side, and right side forming a central fenestration extending along the cage from the leg's front portions to the leg's bottom portions, the cage left leg and right leg first length and the rear leg second length selected so the left leg top portion, right leg top portion, and rear leg top portion may engage sections of the upper bony structure of the two adjacent bony structure and the left leg bottom portion, right leg bottom portion, and rear leg bottom portion may engage sections of the lower bony structure of the two adjacent bony structures when the cage is placed between the two adjacent bony structures; and
an elongated plate extending traverse to the elongated legs through the cage left side and the cage right side, the elongated plate including a first bony fixation element head interface on its right, the first interface extending beyond the cage's right side and a second bony fixation element head interface on its left, the second interface extending beyond the cage's left side, wherein the first interface and the second interface are configured to enable the first bony fixation element and the second bony fixation element to engage the lower bony structure of the two adjacent bony structures when the cage is placed between the two adjacent bony structures.

2. The surgical implant for implantation between two adjacent bony structures of claim 1, wherein the left leg top portion, right leg top portion, and rear leg top portion include protrusions to aid engagement of sections of the upper bony structure of the two adjacent bony structure and the left leg bottom portion, right leg bottom portion, and rear leg bottom portion include protrusions to aid engagement of sections of the lower bony structure of the two adjacent bony structures when the cage is placed between the two adjacent bony structures.

3. The surgical implant for implantation between two adjacent bony structures of claim 1, wherein the two adjacent bony structures are adjacent vertebrae and the cage front left leg and the front right leg first length is selected so the front left leg top portion and the front right leg top portion engage lamina of the upper vertebra of the adjacent vertebrae and the left leg bottom portion and the right leg bottom portion engage lamina of the lower vertebra of the adjacent vertebrae, the rear leg second length is selected so rear leg top portion may engage a spinous process of the upper vertebra of the adjacent vertebrae and the rear leg bottom portion may engage a spinous process of the lower vertebra of the adjacent vertebrae when the cage is placed between the two adjacent vertebrae.

4. The surgical implant for implantation between two adjacent bony structures of claim 3, wherein the elongated plate first interface and the elongated plate second interface are configured to enable a first bony fixation element and a second bony fixation element to engage pedicles of the lower bony vertebra of the two adjacent vertebrae when the cage is placed between the two adjacent vertebrae.

5. The surgical implant for implantation between two adjacent bony structures of claim 3, wherein an inwardly curved front side extending from the front left leg to the right leg along the first length forming a concave section between the left leg and right leg is sized to protect and encase the spinal dural sac between the adjacent vertebrae when the cage is placed between the two adjacent vertebrae.

6. The surgical implant for implantation between two adjacent bony structures of claim 3, wherein the left side extending from the left leg to the rear leg and the right side extending from the right leg to the rear leg from a convex rear section along the elongated rear leg.

7. The surgical implant for implantation between two adjacent bony structures of claim 3, wherein a front top peak is formed on the front side between the front left leg top portion and the front right leg top portion, the front top peak sized to engage lamina of the upper vertebra of the adjacent vertebrae and a front bottom peak is formed on the front side between the front left leg bottom portion and the front right leg bottom portion, the front bottom peak sized to engage lamina of the upper vertebra of the adjacent vertebrae.

8. The surgical implant for implantation between two adjacent bony structures of claim 7, wherein a rear top peak is formed on the rear top portion and sized to engage the spinous process of the upper vertebra of the adjacent vertebrae and a rear bottom peak is formed on the rear bottom portion and sized to engage the spinous process of the lower vertebra of the adjacent vertebrae.

9. The surgical implant for implantation between two adjacent bony structures of claim 1, wherein first length is less than the second length.

10. The surgical implant for implantation between two adjacent bony structures of claim 8, wherein first length is less than the second length and the length from the front top peak to the front bottom peak is less than the length from the rear top peak to the rear bottom peak.

11. A surgical implant for implantation between two adjacent bony structures, the implant including:
a cage including an elongated front left leg extending a first length from a top portion to a bottom portion, a front elongated right leg extending the first length from a top portion to a bottom portion, and an elongated rear leg extending a length from a top portion to a bottom portion, wherein the left leg, right leg, and rear leg form an appropriate triangle when viewed from the respective top portions, an inwardly curved front side extending from the front left leg to the right leg along the first length forming a concave section between the left leg and right leg, a left side extending from the left leg to the rear leg, a right side extending from the right leg to the rear leg, the front side, left side, and right side forming a central fenestration extending along the cage from the leg's front portions to the leg's bottom portions, the cage left leg and right leg first length and the rear leg second length selected so the left leg top portion, right leg top portion, and rear leg top portion may engage sections of the upper bony structure of the two adjacent bony structure and the left leg bottom portion, right leg bottom portion, and rear leg bottom portion may engage sections of the lower bony structure of the two adjacent bony structures when the cage is placed between the two adjacent bony structures; and
a first elongated plate extending parallel to the elongated legs through the cage left side, the first elongated plate including a first bony fixation element head interface on its top, the first interface extending beyond the cage's top left side and a second bony fixation element head interface on its bottom, the second interface extending beyond the cage's bottom left side, wherein the first interface and the second interface are configured to enable the first bony fixation element to engage the upper bony structure of the two adjacent bony structures and the second bony fixation element to engage the lower bony structure of the two adjacent bony structures when the cage is placed between the two adjacent bony structures.

12. The surgical implant for implantation between two adjacent bony structures of claim 11, wherein the left leg top portion, right leg top portion, and rear leg top portion include protrusions to aid engagement of sections of the upper bony structure of the two adjacent bony structure and the left leg bottom portion, right leg bottom portion, and rear leg bottom portion include protrusions to aid engagement of sections of the lower bony structure of the two adjacent bony structures when the cage is placed between the two adjacent bony structures.

13. The surgical implant for implantation between two adjacent bony structures of claim 11, wherein the two adjacent bony structures are adjacent vertebrae and the cage front left leg and the front right leg first length is selected so the front left leg top portion and the front right leg top portion engage lamina of the upper vertebra of the adjacent vertebrae and the left leg bottom portion and the right leg bottom portion engage lamina of the lower vertebra of the adjacent vertebrae, the rear leg second length is selected so rear leg top portion may engage a spinous process of the upper vertebra of the adjacent vertebrae and the rear leg bottom portion may engage a spinous process of the lower vertebra of the adjacent vertebrae when the cage is placed between the two adjacent vertebrae.

14. The surgical implant for implantation between two adjacent bony structures of claim 13, wherein the first elongated plate first interface is configured to enable a first bony fixation element to engage a spinous process of the upper bony vertebra of the two adjacent vertebrae and the first elongated plate second interface is configured to enable a second bony fixation element to engage a spinous process of the lower bony vertebra of the two adjacent vertebrae when the cage is placed between the two adjacent vertebrae.

15. The surgical implant for implantation between two adjacent bony structures of claim 13, wherein an inwardly curved front side extending from the front left leg to the right leg along the first length forming a concave section between the left leg and right leg is sized to protect and encase the spinal dural sac between the adjacent vertebrae when the cage is placed between the two adjacent vertebrae.

16. The surgical implant for implantation between two adjacent bony structures of claim 13, wherein the left side extending from the left leg to the rear leg and the right side extending from the right leg to the rear leg from a convex rear section along the elongated rear leg.

17. The surgical implant for implantation between two adjacent bony structures of claim 13, wherein a front top peak is formed on the front side between the front left leg top portion and the front right leg top portion, the front top peak sized to engage lamina of the upper vertebra of the adjacent vertebrae and a front bottom peak is formed on the front side between the front left leg bottom portion and the front right leg bottom portion, the front bottom peak sized to engage lamina of the upper vertebra of the adjacent vertebrae.

18. The surgical implant for implantation between two adjacent bony structures of claim 17, wherein a rear top peak is formed on the rear top portion and sized to engage the spinous process of the upper vertebra of the adjacent vertebrae and a rear bottom peak is formed on the rear bottom portion and sized to engage the spinous process of the lower vertebra of the adjacent vertebrae.

19. The surgical implant for implantation between two adjacent bony structures of claim 11, wherein first length is less than the second length.

20. The surgical implant for implantation between two adjacent bony structures of claim 18, wherein first length is less than the second length and the length from the front top peak to the front bottom peak is less than the length from the rear top peak to the rear bottom peak.

21. The surgical implant for implantation between two adjacent bony structures of claim 11, further including a second elongated plate extending parallel to the elongated legs through the cage right side, the second elongated plate including a first bony fixation element head interface on its top, the first interface extending beyond the cage's top right side and a second bony fixation element head interface on its bottom, the second interface extending beyond the cage's bottom right side, wherein the first interface and the second interface are configured to enable the first bony fixation element to engage the upper bony structure of the two adjacent bony structures and the second bony fixation element to engage the lower bony structure of the two adjacent bony structures when the cage is placed between the two adjacent bony structures.

22. The surgical implant for implantation between two adjacent bony structures of claim 21, wherein the second elongated plate first interface is configured to enable a first bony fixation element to engage a spinous process of the upper bony vertebra of the two adjacent vertebrae and the second elongated plate second interface is configured to enable a second bony fixation element to engage a spinous process of the lower bony vertebra of the two adjacent vertebrae when the cage is placed between the two adjacent vertebrae.
